(19) United States
(12) Patent Application Publication
(10) Pub. No.: US 2021/0283307 A1
(43) Pub. Date: Sep. 16, 2021

(54) METHOD FOR EXTRACTING EXTRACELLULAR MATRIX USING SUPERCRITICAL FLUID, AND EXTRACELLULAR MATRIX BIOMATERIAL FOR TISSUE REGENERATION PRODUCED THEREBY

(71) Applicant: DOF INC., Seongnam-si, Gyeonggi-do (KR)

(72) Inventors: Yong-Woo SHIN, Seongnam-si, Gyeonggi-do (KR); Kyu-Byung KIM, Gwangju-si, Gyeonggi-do (KR); Seong-Rae NO, Seongnam-si, Gyeonggi-do (KR); Yoon-Hee SEOL, Seongnam-si, Gyeonggi-do (KR); Jee-Hoon SHIN, Seongnam-si, Gyeonggi-do (KR); Hyoung-Soon KIM, Incheon (KR)

(21) Appl. No.: 17/257,541

(22) PCT Filed: Nov. 22, 2019
(86) PCT No.: PCT/KR2019/016189

§ 371 Date: Dec. 31, 2020

(30) Foreign Application Priority Data
Nov. 28, 2018 (KR) 10-2018-0150057

(51) Int. Cl. A61L 27/36; C12N 5/00; C12N 5/077

(52) U.S. Cl. CPC A61L 27/3691; C12N 5/0653; C12N 5/0081; A61L 27/3633

(57) ABSTRACT

Provided is a method for extracting an extracellular matrix with a supercritical fluid, comprising (a) injecting an extracted adipose tissue into a reactor, (b) pressurizing a solvent to prepare a supercritical fluid, and (c) introducing the supercritical fluid into the reactor to decellularize and delipidate the adipose tissue, and thereby extracting an extracellular matrix.

### TECHNICAL FIELD

The present invention relates to a method for extracting an extracellular matrix from adipose tissue with a supercritical fluid; and an extracellular matrix biomaterial for tissue regeneration produced thereby.

### BACKGROUND

Adipose tissue is a type of loose connective tissue composed mainly of adipocytes. In addition to adipocytes, adipose tissue contains the stromal vascular fraction (SVF) of cells including preadipocytes, fibroblasts, vascular endothelial cells and a variety of immune cells.

Adipose tissue has been recognized as a major endocrine organ over the years, as it produces hormones such as leptin, estrogen, and cytokine TNFα. Adipose tissue is roughly classified as white adipose tissue (WAT) and brown adipose tissue (BAT). The formation of adipose tissue appears to be controlled in part by an adipose tissue organ.

Adipose tissue develops in the subcutaneous tissue, in the mesentery, in the retroperitoneum and the like, and its main role is to store energy in the form of lipids, although it also prevents heat dissipation, mechanically protects the organs, or shows active metabolic activity due to the rich vascularity.

Meanwhile, adipose tissue includes the extracellular matrix.

The extracellular matrix is a construct mainly responsible for structural support, etc. in animals, and consists of the interstitial matrix between cells and the basement membrane. The interstitial matrix between cells fills the interstitial space between various cells, and gels of polysaccharides and fibrous proteins fill the interstitial space between cells and help buffering action of the extracellular matrix.

In addition, the extracellular matrix is an important substance that forms the structure of the human body and regulates vital functions. It is secreted from cells and contains various components such as collagen, laminin, elastin, glycosaminoglycan, and fibronectin.

Various biomaterials, such as tissue repair materials and dressing bandages, with the extracellular matrix, have been introduced, but only some components of the extracellular matrix are used in currently commercialized products, and very useful growth factors and proteins are not properly used.

A method for extracting an extracellular matrix containing useful proteins from the animal-derived adipose tissue, including the human adipose tissue, is being studied, but the human adipose tissue extracted has limited availability due to problems, such as difficulties in cell separation and contamination or immune reactions for allotransplantation.

Recently, liposuction has been often performed for cosmetic surgery, so the conditions for acquiring a large amount of human-derived adipose tissue have been established. However, when extracting useful materials of the extracellular matrix and stem cells from such lipoaspirate, the extracted human adipose tissue contains unnecessary components such as blood and lipids and thus is almost discarded.

Thus, there is an urgent need to develop a method for effectively extracting an extracellular matrix, which is a useful component, from adipose tissue to be discarded and enabling allotransplantation.

### DETAILED DESCRIPTION OF THE INVENTION

### Technical Problem

Thus, the present invention provides a method for extracting an extracellular matrix with a supercritical fluid, the method being capable of effectively removing contaminants from the adipose tissue, including the human-derived adipose tissue, through a pretreatment process, and extracting the extracellular matrix through decellularization and delipidation processes using a supercritical fluid, and an extracellular matrix biomaterial for tissue regeneration produced thereby.

However, the present invention is not limited thereto and other objects not mentioned can be clearly understood by those skilled in the art from the following description.

### Technical Solution

In accordance with an exemplary embodiment, the present invention provides a method for extracting an extracellular matrix with a supercritical fluid, which comprises:

(a) injecting an extracted adipose tissue into a reactor;

(b) pressurizing a solvent to prepare a supercritical fluid; and

(c) introducing the supercritical fluid into the reactor to decellularize and delipidate the adipose tissue, and thereby extracting the extracellular matrix.

In addition, the adipose tissue may be animal- or human-derived adipose tissue.

In addition, in pressurizing, the solvent may be pressurized at 200 to 600 bar.

In addition, in step (C), the supercritical fluid may be introduced into the reactor at a flow rate of 18 to 70 mL/min and reacted for 2 to 12 hours at 30 to 35° C.

In accordance with another exemplary embodiment, the present invention provides a method for extracting an extracellular matrix with a supercritical fluid, which comprises:

(1) washing and mincing an adipose tissue to prepare the adipose tissue;

(2) performing a pretreatment by centrifuging the prepared adipose tissue to remove water and lipids;

(3) placing a carbon dioxide cylinder, placing a co-solvent chamber on one side of the carbon dioxide cylinder, pressurizing carbon dioxide discharged from the carbon dioxide cylinder to prepare a supercritical fluid, and then, mixing the co-solvent discharged from the co-solvent chamber with the supercritical fluid to prepare a solvent, and then introducing the solvent, in a preheated state through a preheater, into the reactor in which the pretreated adipose tissue from step (2) is placed, to decellularize and delipidate the adipose tissue; and

(4) discharging the solvent from one side of the reactor, and recovering the extracellular matrix remaining in the reactor.

Also, in step (1), the washing is performed by washing with deionized water, and then, being allowed to stand for 15 to 20 minutes to remove blood from the adipose tissue. The washing may be performed three to five times.

In addition, in step (1), for the mincing, the washed adipose tissue may be subjected to 400 to 500 W ultrasonication for 6 to 12 minutes.

In addition, the ultrasonication step may be performed with a chiller at a range of 2 to 5° C.

In addition, in step (2), the centrifuging may be performed at 4,000 to 10,000 rpm for 15 to 20 minutes, and at 2 to 5° C. to remove water and lipids.

In addition, in step (2), the supercritical fluid may be prepared by pressurizing the solvent at 200 to 600 bar.

In addition, in step (5), the supercritical fluid may be introduced into the reactor at a flow rate of 18 to 70 mL/min and reacted for 2 to 12 hours at 30 to 35° C.

In addition, in step (3), the supercritical fluid may be introduced into the reactor to react with the adipose tissue, and any one selected from the group consisting of ethanol, ether, and propane may be added as a co-solvent.

In addition, the concentration of the co-solvent may be 10 to 25% (v/v).

In accordance with another aspect, the present invention provides an extracellular matrix biomaterial for tissue regeneration, produced by injecting an extracted adipose tissue into a reactor; pressurizing a solvent to prepare a supercritical fluid; introducing the supercritical fluid into the reactor to decellularize and delipidate the adipose tissue, and thereby extracting an extracellular matrix.

### Advantageous Effects

According to the present invention, decellularization and delipidation from the animal adipose tissue, including human adipose tissue, are very effectively performed through an extraction process with a supercritical fluid, and thus, the extracellular matrix having increased biocompatibility can be extracted.

The removal of contaminants such as blood, etc. via the pretreatment of the extracted adipose tissue and the preceding removal of water and lipids can greatly increase the efficiency of the supercritical extraction process.

In addition, the recovered extracellular matrix contains useful proteins, such as growth factors like IGF-1, collagen, fibronectin, etc., in large quantities, and thus can be used as a biomaterial for regeneration in various ways.

It should be understood that the effects of the present invention are not particularly limited to those described above, and the present invention includes all effects that can be deduced from the detailed description of the invention or the configurations of the invention described in the claims.

### BRIEF DESCRIPTION OF THE DRAWINGS

FIG. 1 is a process flow chart of a method for extracting an extracellular matrix with a supercritical fluid, in accordance with an exemplary embodiment of the present invention.

FIG. 2 is a process flow chart of a method for extracting an extracellular matrix with a supercritical fluid, in accordance with another exemplary embodiment of the present invention.

FIG. 3 is a process chart illustrating a configuration of a supercritical extraction device in a method for extracting an extracellular matrix with a supercritical fluid, in accordance with an exemplary embodiment of the present invention.

FIG. 4 is a photograph of the human-derived adipose tissue after washing in a method for extracting an extracellular matrix with a supercritical fluid, in accordance with an exemplary embodiment of the present invention.

FIG. 5 is a photograph of the washed and centrifuged human-derived adipose tissue in a method for extracting an extracellular matrix with a supercritical fluid, in accordance with an exemplary embodiment of the present invention.

FIG. 6 is a photograph showing the SDS PAGE analysis result of the extracellular matrix obtained by supercritical extraction, in a method for extracting an extracellular matrix with a supercritical fluid, in accordance with an exemplary embodiment of the present invention.

FIG. 7 is a graph showing the amount of DNA remaining in the extracellular matrix over the supercritical extraction time, in a method for extracting an extracellular matrix with a supercritical fluid, in accordance with an exemplary embodiment of the present invention.

FIG. 8 is a PCR amplification photograph showing the amount of DNA remaining in the extracellular matrix over the supercritical extraction time, in a method for extracting an extracellular matrix with a supercritical fluid, in accordance with an exemplary embodiment of the present invention.

FIG. 9 is a photograph of the final product in a method for extracting an extracellular matrix with a supercritical fluid, in accordance with an exemplary embodiment of the present invention.

FIG. 10 is a Western blot photograph for laminin in the obtained sample, in a method for extracting an extracellular matrix with a supercritical fluid, in accordance with an exemplary embodiment of the present invention.

FIG. 11 is a Western blot photograph for collagen I in the obtained sample, in a method for extracting an extracellular matrix with a supercritical fluid, in accordance with an exemplary embodiment of the present invention.

FIG. 12 is a Western blot photograph for fibronectin in the obtained sample, in a method for extracting an extracellular matrix with a supercritical fluid, in accordance with an exemplary embodiment of the present invention.

FIG. 13A is the analysis result of the growth factor IGF in the obtained extracellular matrix, in a method for extracting an extracellular matrix with a supercritical fluid, in accordance with an exemplary embodiment of the present invention.

FIG. 13B is the analysis result of the growth factor bFGF in the obtained extracellular matrix, in a method for extracting an extracellular matrix with a supercritical fluid, in accordance with an exemplary embodiment of the present invention.

FIG. 13C is the analysis result of the growth factor VEGF in the obtained extracellular matrix, in a method for extracting an extracellular matrix with a supercritical fluid, in accordance with an exemplary embodiment of the present invention.

FIG. 13D is the analysis result of the growth factor NGF in the obtained extracellular matrix, in a method for extracting an extracellular matrix with a supercritical fluid, in accordance with an exemplary embodiment of the present invention.

FIG. 14 is a photograph showing a wound healing process in a rat treated with an extracellular matrix biomaterial for tissue regeneration, including the extracellular matrix, in accordance with another embodiment of the present invention.

### BEST MODE FOR CARRYING OUT THE INVENTION

The collected sample was placed on a supercritical extraction device equipped with an extraction reactor, and the supercritical extraction was performed.

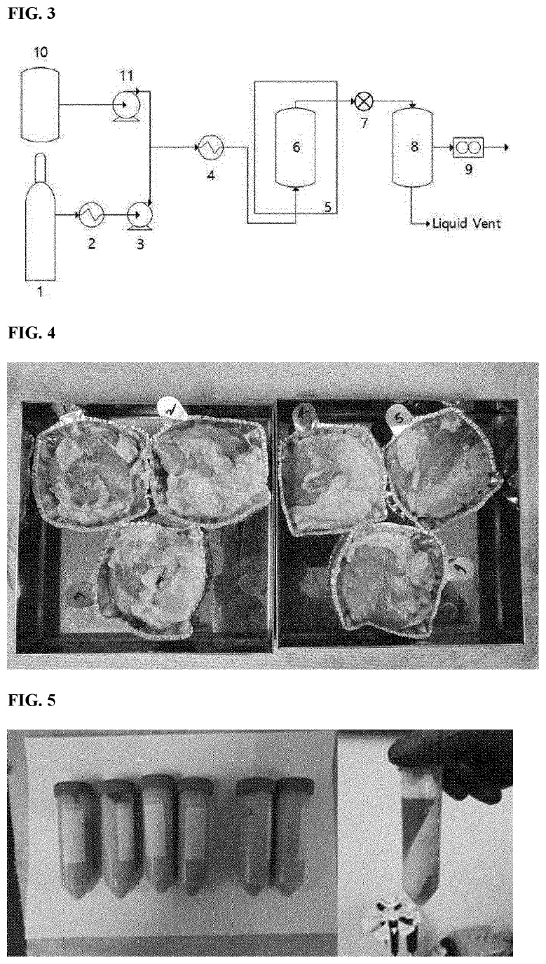

FIG. 3 is a process chart illustrating a configuration of a supercritical extraction device in a method for extracting an extracellular matrix with a supercritical fluid, in accordance with an exemplary embodiment of the present invention.

Referring to FIG. 3, the carbon dioxide cylinder (1) and, along a line to move a solvent, the chiller (2) and the liquid pump (3) are placed on one side of the supercritical extraction device in accordance with an embodiment of the present invention.

The co-solvent chamber (10) is placed on one side of the solvent cylinder (1) and a co-solvent joins the carbon dioxide moving line along the co-solvent pump (11).

The solvent, to which the co-solvent is added, is preheated through the preheater (4), and then introduced into the extraction chamber (6) provided in the oven (5), and penetrates the adipose tissue charged in the extraction chamber (6), and an extraction process with a supercritical fluid proceeds.

The pressure of the extract discharged from one side of the extraction chamber (6) is controlled through the backpressure regulator (7), and the extract is collected in the trap chamber (8).

The flowmeter (9) is provided at one side of the trap chamber (8) to determine the flow rate of the supercritical fluid.

In one embodiment of the present invention, the solvent is carbon dioxide, and the co-solvent is ethanol.

The supercritical fluid was prepared from carbon dioxide by controlling the temperature of the oven (5) at 32° C. and pressurizing the liquid pump (3) at 300 bar, and then the adipose tissue was extracted by introducing the supercritical fluid into the extraction chamber (6) at the flow rate of 20 mL/min.

The extraction reaction was performed for 2 to 12 hours, and after the extraction was completed, a vent of the trap chamber (8) was opened to evaporate and remove the extract, and the delipidated sample was obtained with the supercritical fluid.

### MODE FOR CARRYING OUT THE INVENTION

Hereinafter, preferred embodiments of the present invention will be described in detail with reference to the accompanying drawings.

Advantages and features of the present invention and methods of achieving the advantages and features will be clear with reference to embodiments described in detail below together with the accompanying drawings.

However, the present invention is not limited to embodiments disclosed herein, but will be implemented in various forms. The embodiments are provided so that the present invention is completely disclosed, and a person of ordinary skilled in the art can fully understand the scope of the present invention. Therefore, the present invention will be defined only by the scope of the appended claims.

When it is determined that describing relevant known techniques in detail in describing the present invention can obscure the essence of the present invention, such detailed description will be omitted.

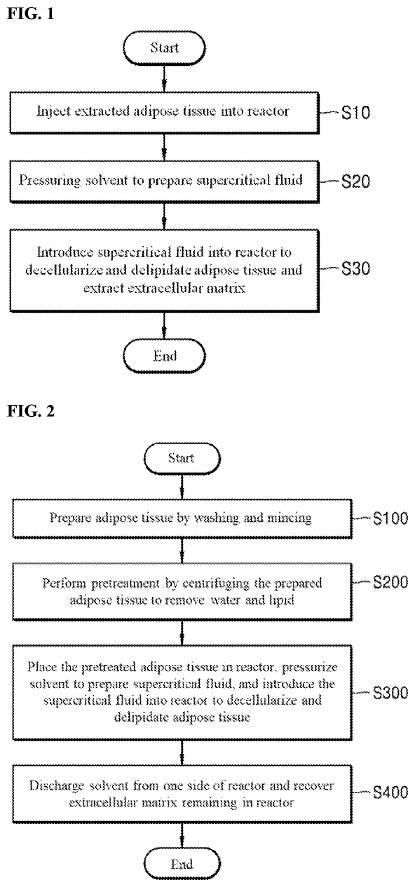

FIG. 1 is a process flow chart of a method for extracting an extracellular matrix with a supercritical fluid, in accordance with an exemplary embodiment of the present invention.

Referring to FIG. 1, the present invention includes: (a) injecting an extracted adipose tissue into a reactor; (b) pressurizing a solvent to prepare a supercritical fluid; and (c) introducing the supercritical fluid into the reactor to decellularize and delipidate the adipose tissue, and thereby extracting the extracellular matrix.

First, the adipose tissue is extracted and injected into the prepared reactor (S10).

The adipose tissue may be animal- or human-derived adipose tissue.

When the adipose tissue is animal-derived adipose tissue, it may be one obtained by washing porcine skin through a known method, immersing in ethyl alcohol, heating in a saline solution, and mincing it.

When the adipose tissue is human-derived adipose tissue, it may be one obtained from the liposuction.

The adipose tissue contains the extracellular matrix in addition to cells, and the extracellular matrix contains genetic materials such as DNA, and useful proteins, such as growth factors, collagen, and fibronectin.

A supercritical fluid is prepared by pressurizing a solvent (S20).

The solvent may be any one selected from the group consisting of carbon dioxide, ammonia, nitrogen, nitrogen monoxide (NO), nitrogen dioxide (NO₂), nitrous oxide (N₂O), sulfur dioxide, hydrogen, water vapor, methane, ethylene, propane, propylene, and a mixed gas thereof, or any one selected from alcohols, including ethanol and methanol, aromatic compounds, including benzene and toluene, but is preferably carbon dioxide.

The carbon dioxide easily reaches the critical point (31.1° C., 73.8 bar), which is a supercritical state, and has a gas-like diffusion characteristic.

Since it has a density similar to that of a liquid, its solubility is high for various compounds.

The density of the supercritical carbon dioxide, which is related to solvent power, can be controlled by changing only the temperature or pressure conditions.

The carbon dioxide at a high density has increased solubility for a non-polar material and thus can dissolve the non-polar material.

For this reason, the supercritical carbon dioxide is preferred as a solvent in chemical reactions, and it is, in particular, preferred in polymerization reactions because it can dissolve various types of monomers.

The supercritical fluid has the advantages that it can save a significant amount of energy because the process of separating the carbon dioxide solvent from the polymer product is possible only with a simple pressure drop; and it is easily separated at room temperature and thus is distinct from the process using an organic solvent in terms of leaving no toxicity in the extract.

In the pressurizing, the solvent may be pressurized at 200 to 600 bar.

The pressurizing converts carbon dioxide to the supercritical fluid. When the pressurizing is performed below the above pressure range, the density of the supercritical fluid is low, and thus the decellularization and delipidation effects on the extracted adipose tissue are reduced. When the pressurizing is performed above the range, the energy due to the pressurizing is unnecessarily consumed, and thus the efficiency of the overall process can be reduced.

The supercritical fluid prepared by pressurizing at 200 bar or more can penetrate the cell wall, effectively dissolve intracellular lipids, and thus delipidate and decellularize the adipose tissue.

When cells are removed from the adipose tissue, the immune response of the obtained extracellular matrix can be inhibited, which is very advantageous for allotransplantation.

The supercritical fluid is introduced into the reactor to decellularize and delipidate the adipose tissue and thus extract the extracellular matrix (S30).

In S30, the supercritical fluid may be introduced into the reactor at a flow rate of 18 to 70 mL/min and reacted for 4 to 12 hours at 30 to 35° C.

The extraction time may be reduced by increasing the flow rate of the supercritical fluid within the above flow rate range.

The extracellular matrix may be obtained by introducing into the reactor the supercritical fluid to react with the adipose tissue within the above range of the reaction time; and decellularizing and delipidating the adipose tissue.

Extracting for less than the extraction time may cause a problem of a decrease in the contents of the growth factors and useful proteins contained in the extracellular matrix. Extracting for more than the extraction time may cause a problem of an unnecessary increase in the processing time although lipids have been already removed.

FIG. 2 is a process flow chart of a method for extracting an extracellular matrix with a supercritical fluid, in accordance with another exemplary embodiment of the present invention.

Referring to FIG. 2, in accordance with another exemplary embodiment, the present invention provides a method for extracting an extracellular matrix with a supercritical fluid, which comprises:

(1) washing and mincing an adipose tissue to prepare the adipose tissue;

(2) performing a pretreatment by centrifuging the prepared adipose tissue to remove water and lipids;

(3) placing a carbon dioxide cylinder, placing a co-solvent chamber on one side of the carbon dioxide cylinder, pressurizing carbon dioxide discharged from the carbon dioxide cylinder to prepare a supercritical fluid, and then, mixing the co-solvent discharged from the co-solvent chamber with the supercritical fluid to prepare a solvent, and then introducing the solvent, in a preheated state through a preheater, into the reactor in which the pretreated adipose tissue from step (2) is placed, to decellularize and delipidate the adipose tissue; and

(4) discharging the solvent from one side of the reactor, and recovering the extracellular matrix remaining in the reactor.

First, the adipose tissue is prepared by washing and mincing (S100).

The adipose tissue may be human-derived adipose tissue.

The human-derived adipose tissue may be one obtained from the liposuction, which is a cosmetic procedure.

The adipose tissue obtained through liposuction contains a large amount of blood due to the procedure. The blood remaining in the adipose tissue has the possibility of continuing to remain during the supercritical extraction process, which may result in inflammation and immune response when the obtained extracellular matrix is used in the allotransplantation.

The washing is performed with deionized water. Then, the adipose tissue is allowed to stand for 15 to 20 minutes to remove blood from the adipose tissue. The washing may be performed three to five times.

The washing involves the addition of deionized water, stirring, and then, allowing the adipose tissue to stand for the above time range, which enables blood to separate from the adipose tissue.

The washing may be performed three to five times. Washing less than three times does not have a blood removal effect.

The washing results in the removal of blood and thus can prevent contamination of the extracellular matrix and immune response.

The mincing may be performed by subjecting the washed adipose tissue to 400 to 500 W ultrasonication for 6 to 12 minutes.

The ultrasound can separate cells from the adipose tissue to promote decellularization and greatly increase a lipid extracting effect in the centrifugation process described later.

The ultrasonication below the above range cannot achieve the decellularization and lipid extraction effects. The ultrasonication above the range can cause the modification of necessary materials such as useful proteins, in addition to the cell separation.

The ultrasonication step may be performed with a chiller at a range of 2 to 5° C.

Since proteins may be modified when heated to 38° C. or higher, controlling the temperature within the above range is particularly preferable because it can prevent the proteins from being modified.

In an embodiment of the present invention, for the ultrasonication, the washed adipose tissue is stirred at 50 rpm or more in a flask to be dispersed and then transferred at a constant flow speed by using a pump to pass through an ultrasonication device. Thus, the ultrasound can penetrate the entire adipose tissue uniformly.

Meanwhile, for the mincing, it is possible to mince the washed adipose tissue with a homogenizer at 6,000 to 18,000 rpm.

When the adipose tissue forms a solid agglomerate, it is preferable to mince the adipose tissue with a homogenizer.

When the speed of the homogenizer is lower than 6,000 rpm, the adipose tissue is not sufficiently minced, which can reduce the supercritical extraction effect. When the speed of the homogenizer is higher than 18,000 rpm, the mincing effect of adipose tissue is not proportionally high, and the overall process efficiency can be decreased.

The washed, minced, and thus prepared adipose tissue may be centrifuged to remove water and lipids (S200).

The centrifuging may be performed at 4,000 to 10,000 rpm for 15 to 20 minutes.

When the rotational speed of the centrifuging is below the above range, the adipose tissue cannot be separated from the water and lipid layers, and the water and lipids remaining in the adipose tissue can result in a problem of reducing the extraction rate of the supercritical extraction step described later.

When the rotational speed of the centrifuging exceeds the above range, the temperature applied to the adipose tissue during the centrifuging process rises too much, or the layer separation effect does not increase anymore to cause a problem of unnecessary energy consumption.

The centrifuging may be performed at 2 to 5° C. to remove water and lipid.

Since proteins can denature at 38° C. or higher, the centrifuging within the above temperature range can prevent the denaturation of proteins and thus is preferable.

Through the centrifuging, water moves to the lower layer, and oil including lipids moves to the upper layer, and thus water and lipids can be separated.

Through the centrifuging, water and lipids are removed from the adipose tissue and thus, the extraction rate of the supercritical extraction process can be increased, and a high purity of the extracellular matrix can be obtained by eliminating contaminants.

Through S100 and S200, contaminants, water, and lipids can be removed from the adipose tissue. When not performing steps S100 and S200, the extraction time increases in the extraction step using a supercritical fluid, and thus, the extraction efficiency can decrease, and the residual contaminants may cause a problem in using the obtained extracellular matrix as a biomaterial.

In particular, when the adipose tissue is human-derived adipose tissue, performing a pretreatment through S100 and S200 is highly preferable.

The pretreatment may further include a heat drying or freeze-drying step after centrifuging the adipose tissue. The drying can remove low-boiling point materials remaining in the adipose tissue after the centrifuging and thus reduce the extraction time in the extraction step with the supercritical fluid.

The pretreated adipose tissue is placed in the reactor, and a solvent is pressurized to prepare a supercritical fluid, and the supercritical fluid is introduced into the reactor to decellularize and delipidate the adipose tissue (S300).

In S300, the supercritical fluid may be prepared by pressurizing the solvent at 200 to 600 bar.

The pressurizing converts carbon dioxide to the supercritical fluid. When the pressurizing is performed below the range, the density of the supercritical fluid is low, and thus the decellularization and delipidation effects on the extracted adipose tissue are reduced. When the pressurizing is performed above the range, the energy due to the pressurizing is unnecessarily consumed, and thus the efficiency of the overall process can be reduced.

The supercritical fluid prepared by pressurizing at 200 bar or more can penetrate the cell wall, and effectively dissolve and remove intracellular lipids, thereby delipidating and decellularizing the adipose tissue.

When cells are removed from the adipose tissue, the immune response of the obtained extracellular matrix can be inhibited, which is very advantageous for allotransplantation.

By delipidating the adipose tissue and allowing useful substances such as proteins to remain therein, the extracellular matrix can be used as various biomaterials.

The supercritical fluid is introduced into the reactor to extract the extracellular matrix by decellularizing and delipidating the adipose tissue.

The supercritical fluid may be introduced into the reactor at a flow rate of 18 to 70 mL/min and reacted for 2 to 12 hours at 30 to 35° C.

The extraction time may be reduced by increasing the flow rate of the supercritical fluid within the above flow rate range.

The extracellular matrix may be obtained by introducing into the reactor the supercritical fluid to react with the adipose tissue within the above reaction time, and decellularizing and delipidating the adipose tissue.

Reacting for less than the above reaction time may cause a problem of a decrease in the contents of growth factors and useful proteins contained in the extracellular matrix. Reacting for more than the above reaction time may cause a problem of an unnecessary increase in the extraction time although lipids have been already removed.

In S400, when the supercritical fluid may be introduced into the reactor to react with the adipose tissue, any one selected from the group consisting of ethanol, ether, and propane may be added as a co-solvent.

The addition of the co-solvent can shorten the extraction time.

Since ethanol has a polarity, it helps to remove lipids and disinfects contaminants to exhibit a sterilization effect, and ether and propane can reduce the lipid removal time.

The concentration of the co-solvent may be 10 to 25% (v/v).

When adding the co-solvent within the above concentration range, the solvent power of the supercritical fluid for lipids can increase, and the protein content of the obtained extracellular matrix may increase.

The solvent is discharged from one side of the reactor, and the extracellular matrix remaining in the reactor is recovered (S400).

When discharging the solvent from one side of the reactor, the supercritical fluid which has dissolved lipids is discharged in all without remaining in the adipose tissue, and thus the contamination problem of the obtained extracellular matrix can be solved. When using an organic solvent, this effect cannot be expected.

In accordance with another aspect, the present invention provides an extracellular matrix biomaterial for tissue regeneration, including the extracted extracellular matrix.

The extracellular matrix biomaterial may be tissue repair materials, dressing bandages, etc., and is not limited thereto as long as its active ingredient contains the extracellular matrix.

Hereinafter, preferred examples will be presented for a better understanding of the present invention. It is to be understood, however, that these examples are for illustrative purposes only and are not intended to limit the scope of the present invention.

### Example 1: Extraction of Extracellular Matrix

(1) Pretreatment

To extract the extracellular matrix from an adipose tissue, the adipose tissue obtained from the liposuction process was subjected to a contamination prevention treatment and transferred. Then, deionized water in an amount visually sufficient to completely wash away the blood contained in the adipose tissue was injected by using a separatory funnel and mixed. Then, the adipose tissue was allowed to stand for 20 minutes. Thereafter, washing was carried out a total of five times.

1000 mL of the washed adipose tissue was placed in a flask, dispersed at 50 rpm with a stirrer, and then passed through the ultrasonication device at a constant flow rate by using a pump (120 rpm pumping). Thus, the ultrasound was allowed to penetrate the entire adipose tissue uniformly.

The ultrasonic power output was controlled at 400 W, and the adipose tissue was minced for 6 minutes. During ultrasonication, the adipose tissue was maintained at 3° C. by using a chiller.

The adipose tissue subjected to ultrasonication was transferred to a centrifuge and centrifuged for 20 minutes at 4,000 rpm. The water in the lower layer and the lipids in the upper layer were separated and removed.

(2) Supercritical Extraction

The collected sample was placed on a supercritical extraction device equipped with an extraction reactor, and the supercritical extraction was performed.

FIG. 3 is a process chart illustrating a configuration of a supercritical extraction device in a method for extracting an extracellular matrix with a supercritical fluid, in accordance with an exemplary embodiment of the present invention.

Referring to FIG. 3, the carbon dioxide cylinder (1) and, along a line to move a solvent, the chiller (2) and the liquid pump (3), are placed on one side of the supercritical extraction device in accordance with an embodiment of the present invention.

The co-solvent chamber (10) is placed on one side of the solvent cylinder (1) and a co-solvent joins the carbon dioxide moving line along the co-solvent pump (11).

The solvent to which the co-solvent is added is preheated through the preheater (4), and then introduced into the extraction chamber (6) provided in the oven (5), and penetrates the adipose tissue charged in the extraction chamber (6), and an extraction process with a supercritical fluid proceeds.

The pressure of the extract discharged from one side of the extraction chamber (6) is controlled through the backpressure regulator (7), and then the extract is collected in the trap chamber (8).

The flowmeter (9) is provided at one side of the trap chamber (8) to determine the flow rate of the supercritical fluid.

In one embodiment of the present invention, the solvent is carbon dioxide, and the co-solvent is ethanol.

The supercritical fluid was prepared from carbon dioxide by controlling the temperature of the oven (5) at 32° C. and pressurizing the liquid pump (3) at 300 bar, and then the adipose tissue was extracted by introducing the supercritical fluid into the extraction chamber at the flow rate of 20 mL/min.

The extraction reaction was performed for 2 to 12 hours, and after the extraction was completed, a vent of the trap chamber (8) was opened to evaporate and remove the extract, and the delipidated sample was obtained with the supercritical fluid.

### Example 2: Preparation of Biomaterial

The extracellular matrix obtained in Example 1 was minced and mixed with a collagen-mixed viscous solution, and then applied to a polyurethane polymer scaffold and dried to prepare an extracellular matrix biomaterial for tissue regeneration.

### Experimental Example 1: Changes Following Pretreatment

FIG. 4 is a photograph of the human-derived adipose tissue after washing in a method for extracting an extracellular matrix with a supercritical fluid, in accordance with an exemplary embodiment of the present invention.

Referring to FIG. 4, it was found that washing with deionized water removed most of the blood and thus reduced the possibility of contamination in the adipose tissue.

FIG. 5 is a photograph of the washed and centrifuged human-derived adipose tissue in a method for extracting an extracellular matrix with a supercritical fluid, in accordance with an exemplary embodiment of the present invention.

Referring to FIG. 5, it was found that centrifuging the adipose tissue caused to form layers so that water and lipids could be removed by centrifugation.

It was found that water and lipids could be removed through washing, mincing, and centrifuging in the pretreatment process.

### Experimental Example 2: Extracellular Matrix Obtained by Supercritical Extraction

The sample obtained through the pretreatment process was introduced into the supercritical extraction device in accordance with Example 2, and the sample obtained after the supercritical extraction was analyzed.

(1) SDS-PAGE Analysis

Whether proteins exist or not before and after the extraction was determined in the extracellular matrix obtained through the supercritical extraction.

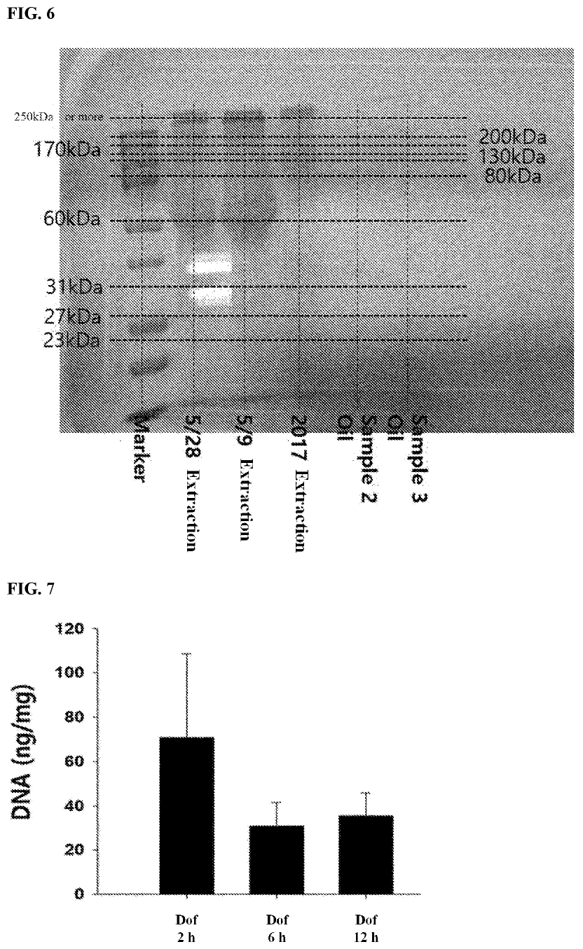

FIG. 6 is a photograph showing the SDS PAGE analysis result of the extracellular matrix obtained by supercritical extraction, in a method for extracting an extracellular matrix with a supercritical fluid, in accordance with an exemplary embodiment of the present invention.

Referring to FIG. 6, it was found that the sample obtained by the supercritical extraction contained proteins of 250 kDa or more when compared to the control group.

Therefore, it was found that the supercritical extraction could make proteins remain in the adipose tissue.

(2) Determination of Total Protein Content

To determine the total protein content in the extracellular matrix depending on the extraction time, the sample was treated with a guanidine solution or a collagenase solution and the total protein content was determined.

TABLE 1

| Extraction time | μg/mg |
|---|---|
| 4 hours | 661.965 |
| 8 hours | 656.5738 |
| 12 hours | 694.687 |
| 12 hours + α hours | 703.2119 |

Table 1 shows the result of the protein content determination in which the sample was treated with a guanidine solution in which 4 M guanidine-HCl, 10 mM EDTA, 50 mM sodium acetate, 65 mM DTT, and a protease inhibitor cocktail were mixed, with varying extraction time. It was found that the sample of 4 to 12 hours all indicated similar protein contents.

TABLE 2

| Extraction time | μg/mg |
|---|---|
| 4 hours | 32.2876 |
| 8 hours | 29.0196 |
| 12 hours | 43.5294 |
| 12 hours + α hours | 74.3791 |

Table 2 shows the result of the protein content determination in which the sample was treated with 0.01% collagenase solution, with varying extraction time. It was found that there was no difference in the protein content between 4 to 12 hours, but the protein content increased with the extraction time of more than 12 hours.

When the extraction time was more than 12 hours, it was found that lipids were mostly removed from the extracellular matrix sample, and thus the protein content per unit mass greatly increased.

(3) Determination of DNA Content

FIG. 7 is a graph showing the amount of DNA remaining in the extracellular matrix over the supercritical extraction time, in a method for extracting an extracellular matrix with a supercritical fluid, in accordance with an exemplary embodiment of the present invention.

TABLE 3

| Extraction time | DNA content (ng/mg) |
|---|---|
| 2 hours | 70.72211 |
| 6 hours | 31.07875 |
| 12 hours | 35.60731294 |

When using the extracted extracellular matrix for allotransplantation, the remaining DNA can act as an antigen to cause the immune response. Thus, it is preferable to control the DNA content at 50 ng/mg or less. FIG. 7 and Table 3 show the DNA content depending on the extraction time.

Referring to FIG. 7 and Table 3, it was found that with the supercritical extraction for 2 hours, the DNA content was about 70 ng/mg, and with the supercritical extraction for 12 hours, the content of the remaining DNA was about 35 ng/mg. Thus, it was found that the content of the remaining DNA was maintained below the reference value.

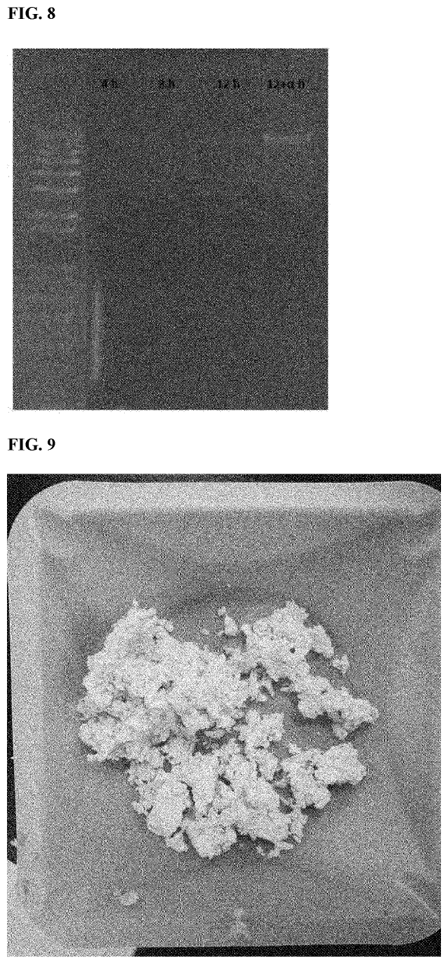

FIG. 8 is a PCR amplification photograph showing the amount of DNA remaining in the extracellular matrix over the supercritical extraction time, in a method for extracting an extracellular matrix with a supercritical fluid, in accordance with an exemplary embodiment of the present invention.

Referring to FIG. 8, a clear band was not found at the extraction time of 2 to 12 hours, which indicates that the DNA content did not increase significantly within the above range of the extraction time.

It was found that even when recovering proteins by concentrating through the supercritical extraction process, the content of the remaining DNA did not significantly increase and the extracellular matrix having the DNA content below the reference value at which the immune response can be inhibited could be obtained.

(4) Content of Useful Proteins

It was determined whether useful proteins contained in the extracellular matrix remain after pretreatment and supercritical extraction.

FIG. 9 is a photograph of the final product in a method for extracting an extracellular matrix with a supercritical fluid, in accordance with an exemplary embodiment of the present invention.

Referring to FIG. 9, it was found that water and lipids were removed following the supercritical extraction, and thus the volume of the adipose tissue decreased, and the adipose tissue was solidified.

TABLE 4

| Extraction time | μg/mg |
|---|---|
| 4 hours | 1.6846 |
| 8 hours | 2.8030 |
| 12 hours | 8.5931 |
| 12 hours + α hours | 39.1225 |

Table 4 shows the elastin content in the obtained extracellular matrix depending on the supercritical extraction time. The content of elastin, which is a useful protein, increased at 4 to 12 hours. It was found that when the extraction time was longer than 12 hours, lipids were mostly removed, and thus the protein per unit mass was enriched, and the content increased sharply.

TABLE 5

| Extraction time | μg/mg |
|---|---|
| 4 hours | 13.1782 |
| 12 hours | 27.2586 |
| 12 hours + α hours | 122.7759 |

Table 5 shows the collagen content in the obtained extracellular matrix depending on the supercritical extraction time. As the extraction time increased from 4 to 12 hours, the collagen content also increased. When the extraction time was longer than 12 hours, the collagen content increased sharply, and thus, it was found that the protein was enriched.

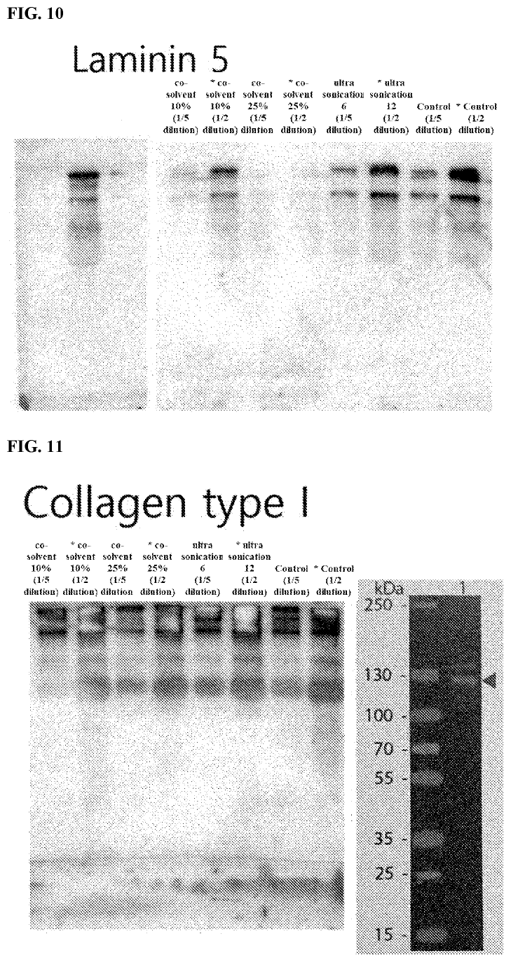

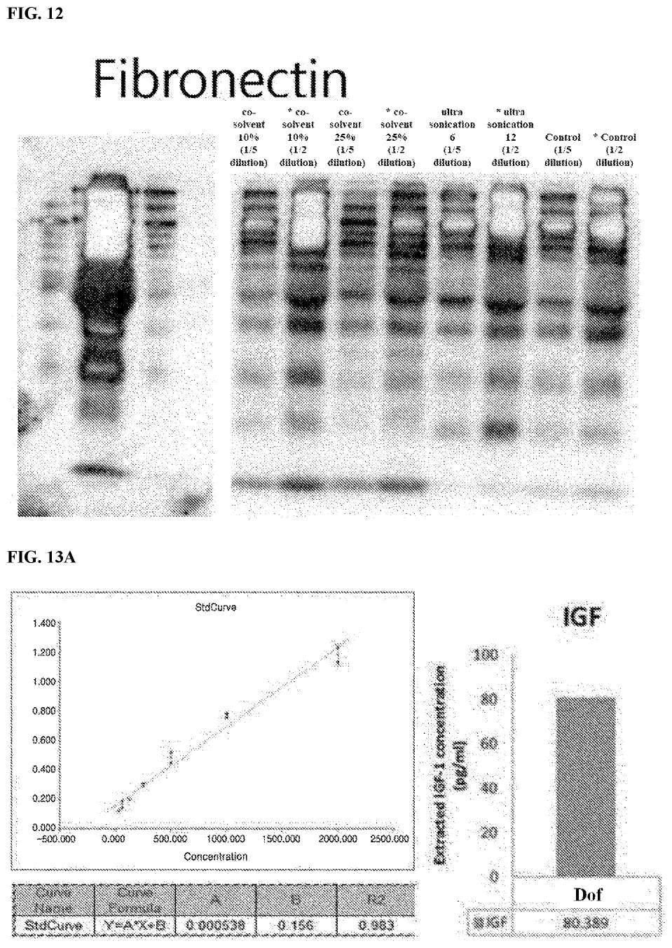

FIG. 10 is a Western blot photograph for laminin of the obtained sample, in a method for extracting an extracellular matrix with a supercritical fluid, in accordance with an exemplary embodiment of the present invention; FIG. 11 is a Western blot photograph for collagen I of the obtained sample, in a method for extracting an extracellular matrix with a supercritical fluid, in accordance with an exemplary embodiment of the present invention; and FIG. 12 is a Western blot photograph for fibronectin of the obtained sample, in a method for extracting an extracellular matrix with a supercritical fluid, in accordance with an exemplary embodiment of the present invention.

Referring to FIGS. 10 to 12, it was found that the sample after ultrasonication contained the useful proteins, compared with the control groups without pretreatment and supercritical extraction. It was also found that even when increasing the concentration of the co-solvent up to 25%, the proteins remained.

It was found that the ethanol co-solvent increased the sterilization effect as the concentration thereof increased, thereby preventing contamination while maintaining the protein content.

(5) Determination of the Growth Factor Content

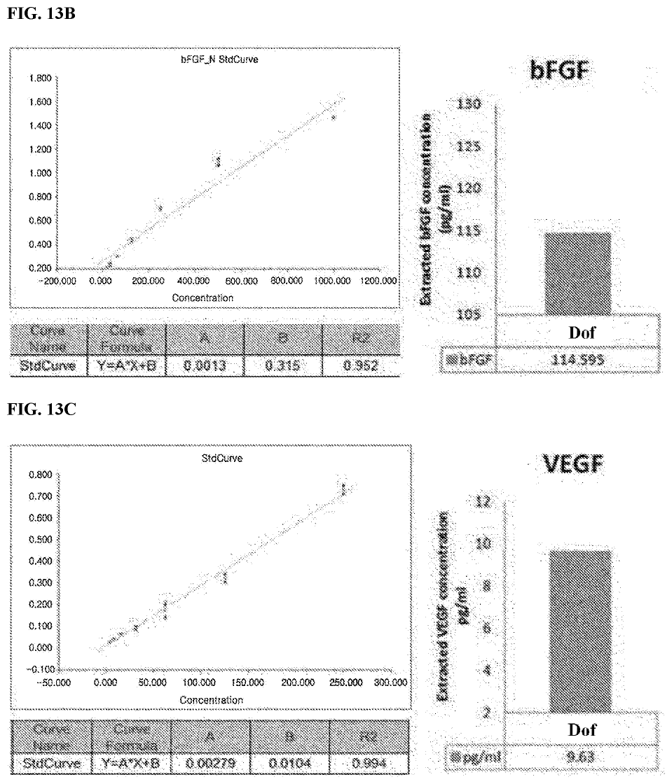

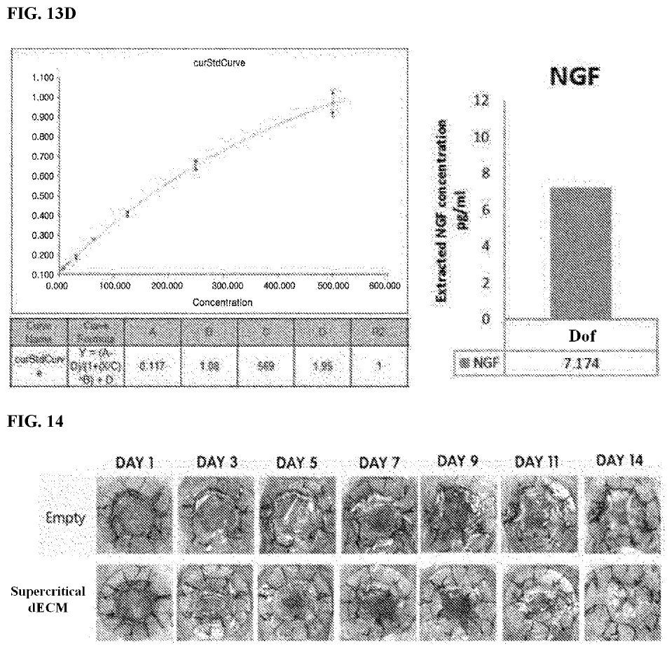

FIGS. 13A to 13D are the analysis results of the growth factors in the obtained extracellular matrix, in a method for extracting an extracellular matrix with a supercritical fluid, in accordance with an exemplary embodiment of the present invention.

Referring to FIGS. 13A to 13D, the growth factors bFGF, IGF, NGF, and VEGF were identified in the extracellular matrix obtained through pretreatment and 4-hour supercritical extraction. The contents of bFGF, IGF, NGF, and VEGF were found to be 114.595 pg/mL, 80.389 pg/mL, 7.174 pg/mL, and 9.63 pg/mL, respectively.

Therefore, it was found that the growth factors remained even after the pretreatment process through washing and mincing and the lipid removal through supercritical extraction.

### Experimental Example 3: Wound Repair with an Extracellular Matrix-Containing Dressing Bandage

The wound repair effect was determined with a dressing bandage in which the extracellular matrix was applied to a polymer scaffold.

6-week-old male Sprague-Dawley (SD) rats were depilated on the back of each test group using an animal electric clipper. Then, the epidermis of skin was disinfected with iodine tincture and a 20 mm circular defect window was created on the skin on both sides of the back at 2 cm outward from the lumbar spine, with surgical scalpel blade no. 11 and thus a wound was induced.

TABLE 6

| Period (day) | Wound |
|---|---|
| 1 | No change |
| 5 | Epidermis repair |
| 14 | Dermis repair |

FIG. 14 is a photograph showing a wound healing process of a rat treated with an extracellular matrix biomaterial for tissue regeneration, including the extracellular matrix in accordance with another embodiment of the present invention. Referring to Table 6 and FIG. 14, it was found that compared to the control group not treated with the biomaterial, the wound healing recovery rate was increased in the experimental group treated with the biomaterial of Example 2. In particular, after five days, it was found that the epidermis repair proceeded. Thus, it was confirmed that the biomaterial of the present invention had an excellent wound repair effect compared to commercially available nitric oxide-based dressing bandages.

Therefore, the present invention relating to a method for extracting an extracellular matrix with a supercritical fluid and an extracellular matrix biomaterial for tissue regeneration produced thereby can, through washing, prevent contamination of animal- or human-derived adipose tissue, and through ultrasonication and centrifugation, significantly shorten the extraction time of the lipid extraction step with a supercritical fluid, and thus increase the efficiency of the method for extracting an extracellular matrix.

Further, it was found that the extracellular matrix obtained through extraction with a supercritical fluid for more than a certain extraction time had a limited content of DNA and thus could inhibit the immune response upon allotransplantation, and contained large amounts of useful proteins and contained growth factors intactly. Therefore, it was found that only unnecessary water and lipids were removed from the adipose tissue through the pretreatment and supercritical extraction processes, and thus, the extracellular matrix, which was the target material, could be obtained very effectively.

In addition, the optimal pressure condition of the supercritical fluid solvent which could show the decellularization and delipidation effects on the human-derived adipose tissue was identified, thereby significantly increasing the efficiency of the supercritical fluid extraction method.

While the specific exemplary embodiments related with the method for extracting an extracellular matrix from adipose tissue with the supercritical fluid and the extracellular matrix biomaterial for tissue regeneration produced thereby in accordance with the present invention have been described above, it is obvious that the exemplary embodiments may be modified to various exemplary embodiments without departing from the scope of the present invention.

Therefore, the scope of the present invention should not be limited to the described exemplary embodiments, but should be defined by the claims to be described below and the equivalents of the claims.

That is, it should be understood that the aforementioned exemplary embodiments are described for illustration in all aspects and are not limited, and the scope of the present invention shall be represented by the claims to be described below, instead of the detailed description, and it should be construed that all of the changes or modified forms induced from the meaning and the scope of the claims, and an equivalent concept thereto are included in the scope of the present invention.

### DESCRIPTION OF REFERENCE NUMERALS

1: Carbon dioxide cylinder 2: Chiller
3: Liquid pump 4: Preheater
5: Oven 6: Extraction chamber
7: Backpressure regulator 8: Trap chamber
9: Flowmeter 10: Co-solvent chamber
11: Co-solvent pump

1. A method for extracting an extracellular matrix with a supercritical fluid, comprising:
(a) injecting an extracted adipose tissue into a reactor;
(b) pressurizing a solvent to prepare a supercritical fluid; and
(c) introducing the supercritical fluid into the reactor to decellularize and delipidate the adipose tissue, and thereby extracting the extracellular matrix,
wherein in the pressurizing of step (b), the solvent is pressurized at 200 to 600 bar, and
wherein in step (c), the supercritical fluid is introduced into the reactor at a flow rate of 18 to 70 mL/min and reacted for 2 to 12 hours at 30 to 35° C.

2. The method of claim 1, wherein the adipose tissue is animal- or human-derived adipose tissue.

3.-4. (canceled)

5. A method for extracting an extracellular matrix with a supercritical fluid, comprising:
(1) washing and mincing an adipose tissue to prepare the adipose tissue;
(2) performing a pretreatment by centrifuging the prepared adipose tissue to remove water and lipids;
(3) placing a carbon dioxide cylinder, placing a co-solvent chamber on one side of the carbon dioxide cylinder, pressurizing carbon dioxide discharged from the carbon dioxide cylinder to prepare a supercritical fluid, and then, mixing the co-solvent discharged from the co-solvent chamber with the supercritical fluid to prepare a solvent, and then introducing the solvent, in a preheated state through a preheater, into a reactor in which the pretreated adipose tissue from step (2) is placed, to decellularize and delipidate the adipose tissue; and
(4) discharging the solvent from one side of the reactor, and recovering the extracellular matrix remaining in the reactor.

6. The method of claim 5, wherein in step (1), the washing is performed with deionized water, and then, the adipose tissue is allowed to stand for 15 to 20 minutes to remove blood, and the washing is performed three to five times.

7. The method of claim 5, wherein in step (1), the mincing further comprises subjecting the washed adipose tissue to 400 to 500 W ultrasonication for 6 to 12 minutes.

8. The method of claim 7, wherein the ultrasonication step is controlled with a chiller in a range of 2 to 5° C.

9. The method of claim 5, wherein in step (2), the centrifuging is performed at 4,000 to 10,000 rpm for 15 to 20 minutes, and at 2 to 5° C. to remove water and lipids.

10. The method of claim 5, wherein in step (3), the supercritical fluid is prepared by pressurizing the solvent at 200 to 600 bar.

11. The method of claim 5, wherein in step (3), the supercritical fluid is introduced into the reactor at a flow rate of 18 to 70 mL/min and reacted for 2 to 12 hours at 30 to 35° C.

12. The method of claim 5, wherein in step (3), the supercritical fluid is introduced into the reactor to react with the adipose tissue, and any one selected from the group consisting of ethanol, ether, and propane is added as a co-solvent.

13. The method of claim 12, wherein the concentration of the co-solvent is 10 to 25% (v/v).

14. An extracellular matrix biomaterial for tissue regeneration, produced by injecting an extracted adipose tissue into a reactor; pressurizing a solvent to prepare a supercritical fluid; introducing the supercritical fluid into the reactor to decellularize and delipidate the adipose tissue, and thereby extracting an extracellular matrix.
